(19) United States
(12) Patent Application Publication
(10) Pub. No.: US 2011/0229564 A1
(43) Pub. Date: Sep. 22, 2011

(54) Pharmaceutical Compositions Of Carvedilol Salts And Process For Preparation Thereof

(75) Inventors: Jatin Desai, Hauppauge, NY (US); Vipul Rasikbhai Gajjar, Gujarat (IN); Gaurav Navinbhai Mistry, Gujarat (IN); Parva Yogeshchandra Purohit, Gujarat (IN)

(73) Assignee: Amneal Pharmaceuticals, L.L.C., Bridgewater, NJ (US)

(21) Appl. No.: 13/051,212

(22) Filed: Mar. 18, 2011

(30) Foreign Application Priority Data
Mar. 22, 2010 (IN) 0293/KOL/2010

(51) Int. Cl. A61K 9/52; A61K 9/00; A61K 31/403; A61K 9/48; B05D 3/00; A61P 9/12

(52) U.S. Cl. 424/456; 424/400; 514/411; 424/463; 424/457; 427/2.14; 427/2.21

(57) ABSTRACT

The present invention relates provides pharmaceutical compositions comprising an amorphous carvedilol salt and one or more pharmaceutically acceptable excipients, wherein the amorphous carvedilol salt is formed in situ during the preparation of the pharmaceutical composition. The amorphous carvedilol salt is preferably an amorphous carvedilol phosphate salt. The pharmaceutical compositions can be prepared by providing one or more inert cores; contacting the core or cores with a solution or a dispersion comprising carvedilol, an acid component and optionally a binder, in a solvent; removing the solvent; and optionally coating the core or cores with an extended release composition. Preferred pharmaceutical compositions contain both immediate-release pellets and at least one population of extended-release pellet.

### CROSS-REFERENCE TO RELATED APPLICATIONS

This application claims priority under 35 U.S.C. §119(a) to Indian Patent Application No. 0293/KOL/2010, filed on Mar. 22, 2010, the entire content of which is hereby incorporated by reference in its entirety.

### FIELD OF THE INVENTION

Aspects of the present invention relate to pharmaceutical compositions comprising carvedilol salts. Aspects of the present invention also relate to extended release pharmaceutical compositions comprising carvedilol salts. Further aspects of the present invention relate to processes for preparing such compositions. Embodiments include amorphous forms of carvedilol salts, including carvedilol phosphate.

### BACKGROUND OF THE INVENTION

Carvedilol is disclosed in U.S. Pat. No. 4,503,067 (assigned to Boehringer Mannheim, GmbH, Germany) and is chemically known as (±)-1-(9H-carbazol-4-yloxy)-3-[[2(2-methoxyphenoxy)ethyl]amino]-2-propanol. Carvedilol is a racemic mixture of R(+) and S(−) enantiomers, represented by structural Formula I below.

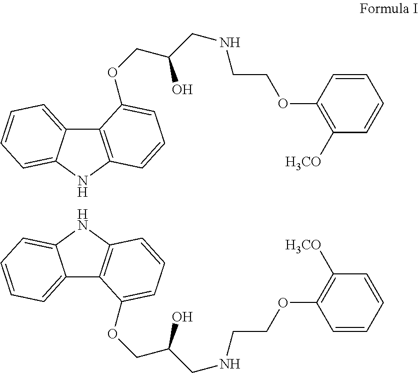

Both carvedilol enantiomers are nonselective β-adrenergic blocking agents with α₁-blocking activity, while S(−) enantiomer also has non-selective β-adrenoreceptor blocking activity. Carvedilol is used for treatment of hypertension and congestive heart failure and is the active ingredient in GSK's COREG®.

There are many known polymorphic and pseudopolymorphic forms of carvedilol. For instance, WO 1999/05105, WO 2002/00216, WO 2003/059807 and WO 2006/135757 describe Forms I to VI of crystalline forms of carvedilol. Likewise, US 2006/0148878 discloses various pseudopolymorphic forms of carvedilol.

At pH values in the pharmaceutically relevant range of 1 to 8, the solubility of carvedilol in aqueous media ranges from about 0.01 mg/ml to about 1 mg/ml. A drug needs to be in solution if it is to pass from the intestine into systemic circulation, and it is generally accepted that where aqueous solubility is less than 5 mg/ml, absorption following administration of an oral dose can be problematic. Furthermore, carvedilol is subject to degradation, forming various unwanted degradation products. Thus, carvedilol has solubility and stability problems which indicate that its bioavailability is low.

In the field of pharmaceutical formulation manufacturing, the problems as described above may be overcome by choice of an appropriate salt form of the drug. It is essential to have a form of drug that has sufficient water solubility to ensure good in vivo absorption. For example, carvedilol exhibits reduced solubility as its hydrochloride salt, which is the protonated form that would be generated in an acidic medium such as gastric fluid. In this regard, the solubility characteristics of the crystalline carvedilol phosphate taught in WO 2004/002419, US 2005/0169994 and US 2006/0182804 are purportedly superior.

There are several patents and patent applications that are directed to salts of carvedilol, and also to their preparation. For example, U.S. Pat. No. 4,503,067 describes salts of carvedilol with acids such as hydrochloric acid, hydrobromic acid, phosphoric acid, sulphuric acid, acetic acid, citric acid, maleic acid or benzoic acid. WO 2004/002419 discloses crystalline carvedilol dihydrogen phosphate hemihydrate, carvedilol dihydrogen phosphate dihydrate, carvedilol dihydrogen phosphate, carvedilol dihydrogen phosphate methanol solvate and carvedilol hydrogen phosphate. The various polymorphic forms of carvedilol phosphate may differ in physical properties such as bulk density, particle size, aqueous solubility, chemical stability, and other physico-chemical properties. Further, WO 2008/002683 discloses an amorphous form of carvedilol phosphate, process for preparing amorphous form, and the use of amorphous form in the preparation of pharmaceutical compositions. It also discloses that the amorphous form may have increased solubility and/or bioavailability than their crystalline counterparts, and thus may be more desirable for pharmaceutical purposes.

There is a clinical rationale for long-term treatment of hypertension with carvedilol and accordingly it would be beneficial to provide a controlled release composition, wherein carvedilol is more completely released from the dosage form. Furthermore, a controlled release composition offers a reduced standard deviation of the concentrations of carvedilol in plasma after administration, which gives rise to a more predictable concentration of carvedilol in plasma. Also, a dose regimen with lower frequency of administration will potentially improve patient compliance. In light of the foregoing, a salt form of carvedilol, such as carvedilol phosphate, with greater aqueous solubility, chemical stability, etc., may offer potential benefits for provision of medicinal products containing the drug carvedilol, including the ability to achieve desired or prolonged systemic drug levels by sustaining absorption along the gastro-intestinal tract, particularly in regions of neutral pH where carvedilol has minimal solubility. In this regard, carvedilol phosphate is the active ingredient in GSK's COREG® CR extended release capsules.

There exists a need in the art for alternate ways to formulate compositions of carvedilol salts especially extended release compositions. We have found that robust compositions comprising carvedilol salts may be prepared by obtaining carvedilol salt in situ from carvedilol base in the process of preparing the pharmaceutical composition thereof.

### SUMMARY OF THE INVENTION

Aspects of the present invention relate to pharmaceutical compositions comprising carvedilol salts.

One aspect of the present invention relates to a pharmaceutical composition comprising an amorphous carvedilol salt and one or more pharmaceutically acceptable excipients, wherein the amorphous carvedilol salt is formed in situ during the preparation of the pharmaceutical composition. In one or more preferred embodiments, the amorphous carvedilol salt is an amorphous carvedilol phosphate salt.

In one or more embodiments, the pharmaceutical composition comprises:

a. one or more cores comprising an amorphous carvedilol salt; and

b. optionally a film coating surrounding the core or cores.

In one or more additional embodiments, the pharmaceutical composition comprises:

a. one or more inert cores; and

b. a coating on the inert core or cores comprising an amorphous carvedilol salt.

Another aspect of the present invention relates to an extended release pharmaceutical composition comprising an amorphous carvedilol salt and one or more pharmaceutically acceptable excipients, wherein the amorphous carvedilol salt is formed in situ during the preparation of the pharmaceutical composition.

In one or more embodiments, the extended release pharmaceutical composition comprises:

a. one or more cores comprising an amorphous carvedilol salt; and

b. an extended release polymer coat surrounding the core or cores.

In one or more additional embodiments, the extended release pharmaceutical composition comprises:

a. one or more inert cores;

b. a first coating on the inert core or cores comprising an amorphous carvedilol salt; and

c. a second coating on the first coating or coatings comprising an extended release polymer.

Another aspect of the present invention relates to a process for the preparation of a pharmaceutical composition comprising an amorphous carvedilol salt formed in situ.

In one or more embodiments, the process comprises:

a. providing a solution or dispersion comprising carvedilol, an acid component and optionally a binder;

b. contacting the solution or dispersion with at least one pharmaceutically acceptable excipient;

c. processing the product of step b. to obtain one or more cores; and

d. optionally coating the core or cores with a film coating composition.

In one or more additional embodiments, the process comprises:

a. providing one or more inert cores;

b. contacting the inert core or cores with a solution or a dispersion comprising carvedilol, an acid component and optionally a binder, in a solvent; and

c. removing the solvent.

Another aspect of the present invention relates to a process for the preparation of an extended release pharmaceutical composition comprising an amorphous carvedilol salt formed in situ.

In one or more embodiments, the process comprises:

a. providing a solution or dispersion comprising carvedilol, an acid component and optionally a binder;

b. contacting the solution or dispersion with at least one pharmaceutically acceptable excipient;

c. processing the product of step b. to obtain one or more cores; and

d. coating the core or cores with an extended release composition.

In one or more additional embodiments, the process comprises:

a. providing one or more inert cores;

b. contacting the core or cores with a solution or a dispersion comprising carvedilol, an acid component and optionally a binder, in a solvent;

c. removing the solvent; and

d. coating the core or cores with an extended release composition.

The coated cores can be tableted or filled into capsules of appropriate size. In one or more preferred embodiments, capsules contain immediate release cores and at least one population of extended release cores. In one or more additionally preferred embodiments, capsules contain two different populations of extended release cores.

### DETAILED DESCRIPTION

Aspects of the present invention relate to pharmaceutical compositions comprising carvedilol salts.

In accordance with one aspect, the present invention provides a pharmaceutical composition comprising an amorphous carvedilol salt and one or more pharmaceutically acceptable excipients. The carvedilol salt is formed in situ from carvedilol base during the process of preparation of the composition. The compositions so prepared provide significant simplification of the manufacturing operations. Such in situ formation also results in reduced solid and solvent wastage in the manufacturing process.

In one or more embodiments, the pharmaceutical composition comprises:

a. one or mores cores comprising an amorphous carvedilol salt; and

b. optionally a film coating surrounding the core or cores.

In one or more additional embodiments, the pharmaceutical composition comprises:

a. one or more inert cores; and

b. a coating on the inert core or cores comprising an amorphous carvedilol salt.

In accordance with another aspect, the present invention provides an extended release pharmaceutical composition comprising an amorphous carvedilol salt and one or more pharmaceutically acceptable excipients, wherein the amorphous carvedilol salt is formed in situ during the preparation of the pharmaceutical composition.

In one or more embodiments, the extended release pharmaceutical composition comprises:

a. one or more cores comprising an amorphous carvedilol salt; and

b. an extended release polymer coat surrounding the core or cores.

In one or more additional embodiments, the extended release pharmaceutical composition comprises:

a. one or more inert cores;

b. a first coating on the inert core or cores comprising an amorphous carvedilol salt; and

c. a second coating on the first coating or coatings comprising an extended release polymer.

The amorphous carvedilol salt can be any pharmaceutically acceptable salt capable of being formed in situ. Non-limiting examples include carvedilol phosphate, carvedilol citrate, carvedilol malate, carvedilol succinate, carvedilol tartrate, carvedilol fumarate, carvedilol salicylate and the like, with the preferred salt being carvedilol phosphate.

The pharmaceutical compositions of the present invention can be any form suitable for oral administration, such as, for example, tablets and capsules.

In some embodiments, the drug cores of the pharmaceutical compositions have moisture contents less than about 3% by weight, or about 2% by weight, or about 1% by weight, or about 0.5% by weight, or about 0.1% by weight, or less, as measured by, for example, a Karl Fischer method.

In some embodiments, the amorphous carvedilol salt formed in situ in the pharmaceutical compositions is stable at 40° C. and 75% RH for at least 1 month, or 2 months, or 3 months, or more. By stable it is meant that less than about 90%, or about 95%, or about 99%, or about 99.5%, or about 99.9%, or more, of the amorphous carvedilol salt in the pharmaceutical composition converts to one or more crystalline forms as measured by conventional techniques.

In some embodiments, the pharmaceutical compositions have contents of organic volatile impurities less than about 2000 ppm, or about 1000 ppm, or about 500 ppm, or less.

Aspects of the present invention also relate to the preparation of pharmaceutical compositions comprising carvedilol salts. In accordance with one aspect, the present invention provides a process for the preparation of a pharmaceutical composition comprising an amorphous carvedilol salt formed in situ. Such in situ formation results in reduced solid and solvent wastage in the manufacturing process.

In one or more embodiments, the process comprises:

a. providing a solution or dispersion comprising carvedilol, an acid component and optionally a binder;

b. contacting the solution or dispersion with at least one pharmaceutically acceptable excipient;

c. processing the product of step b. to obtain one or more cores; and

d. optionally coating the core or cores with a film coating composition.

In one or more additional embodiments, the process comprises:

a. providing one or more inert cores;

b. contacting the inert core or cores with a solution or a dispersion comprising carvedilol, an acid component and optionally a binder, in a solvent; and

c. removing the solvent.

In accordance with another aspect, the present invention provides a process for the preparation of an extended release pharmaceutical composition comprising an amorphous carvedilol salt formed in situ.

In one or more embodiments, the process comprises:

a. providing a solution or dispersion comprising carvedilol, an acid component and optionally a binder;

b. contacting the solution or dispersion with at least one pharmaceutically acceptable excipient;

c. processing the product of step b. to obtain one or more cores; and

d. coating the core or cores with an extended release composition.

In one or more additional embodiments, the process comprises:

a. providing one or more inert cores;

b. contacting the core or cores with a solution or a dispersion comprising carvedilol, an acid component and optionally a binder, in a solvent;

c. removing the solvent; and

d. coating the core or cores with an extended release composition.

Suitable forms of carvedilol include amorphous or crystalline carvedilol base.

Suitable acid components include organic and inorganic weak acids having pKa values between about 2 and 5, including, without limitation, salicylic acid, citric acid, phosphoric acid, malic acid, succinic acid, tartric acid and fumaric acid.

Suitable solvents include aqueous and organic solvents including, without limitation, water, ethyl acetate, dichloromethane, methylene chloride, and alcohols, such as methanol, ethanol and isopropanol, and the like, and mixtures thereof. The ability to use aqueous solvents may reduce the levels organic volatile impurities in the final product.

The inert cores as used herein may be selected from inert non-pareils conventionally used in pharmaceutical industry. The inert non-pareils may be a pharmaceutically acceptable excipient such as starch, sugar, microcrystalline cellulose, vegetable gums, waxes, silicon dioxide, hydroxypropylmethylcellulose, and the like. The size of the inert non-pareils may vary from about 0.1 mm to about 2 mm. The core may be present in an amount ranging from about 10% to about 90% by weight of the composition.

The term “extended release” as used herein denotes slow release of carvedilol salt over an extended period of time, and includes prolonged, controlled, extended and delayed release profiles. Extended release compositions will generally include an extended release polymer selected from one or more of water-insoluble polymers or water-soluble polymers, and combinations thereof. The water-insoluble polymers may be selected from ammonio methacrylate copolymers (e.g., Eudragit RL and RS), ethyl acrylate-methyl methacrylate co-polymer (e.g., Eudragit NE; Eudragit L30D55); cellulose acetate, ethylcellulose, polyvinyl alcohol, and the like, and mixtures thereof. Water-soluble polymers may be selected from hydroxypropyl methylcellulose, alginates, xanthan gum, polyethylene oxide, and the like, and combinations thereof. The extended release polymer may be present in an amount ranging from about 1% to about 30% by weight of the composition. A preferred group of extended-release polymers is the Eudragit series of polymers, both water-insoluble, pH-independent (e.g., Eudragit RL and RS and Eudragit NE) and water-soluble, pH dependent (e.g., Eudragit L30D55). A particularly preferred extended-release polymer is Eudragit L30D55, which provides dissolution above about pH 5.5.

The pharmaceutical compositions as described herein are preferably for oral delivery in the form of capsules or tablets and may comprise one or more pharmaceutically acceptable excipients, such as, for example, diluents, binders, disintegrants, surfactants, lubricants, plasticizers, anti-tacking agents, opacifiers, coloring agents, pore-forming agents, and the like.

Diluents suitable for use in the present invention, include, but are not limited to, sugars such as lactose, sucrose, dextrose, and the like; microcrystalline cellulose, sugar alcohols such as mannitol, sorbitol, xylitol, calcium carbonate, dicalcium phosphate, tribasic calcium phosphate, calcium sulphate, magnesium carbonate, starch, and the like, and combinations thereof. The diluent(s) may be present in an amount ranging from about 1% to about 25% by weight of the composition.

Binders suitable for use in the present invention, include, but are not limited to, polyvinylpyrrolidone, copovidone, hydroxypropyl cellulose, hydroxypropyl methylcellulose, starch, sodium alginate, gums, and the like, and combinations thereof. The binder(s) may be present in an amount ranging from about 1% to about 15% by weight of the composition.

Disintegrants suitable for use in the present invention, include, but are not limited to, croscarmellose sodium, crospovidone, sodium starch glycolate, pregelatinized starch, and the like, and combinations thereof. The disintegrants (s) may be present in an amount ranging from about 0.1% to about 10% by weight of the composition.

Surfactants suitable for use in the present invention, include, but are not limited to sorbitan derivatives (such as Tween™, Span™), mono-, di- and polyglycerides, sugar derivatives (sucrose mono- and distearates), polyethylene glycol esters and ethers, polyethylene and polypropylene glycol block copolymers (such as Pluronic™, Poloxamer™), polyethoxylated oils (such as Cremophor™), sodium lauryl sulfate, and the like, and combinations thereof. The surfactant(s) may be present in an amount ranging from about 0% to about 2% by weight of the composition.

Lubricants and/or anti-tacking agents suitable for use in the present invention, include, but are not limited to talc, magnesium stearate, zinc stearate, calcium stearate, sodium stearyl fumarate, stearic acid, colloidal silicon dioxide, and the like, and combinations thereof. The lubricant(s) and/or anti-tacking agent(s) may be present in an amount ranging from about 0.1% to about 5% by weight of the composition.

Plasticizers suitable for use in the present invention, include, but are not limited to acetyl tributyl citrate, acetyl triethyl citrate, acetylated fatty acid glycerides, castor oil, diethyl phthalate, diethyl sebacate, dibutyl sebacate, dimethyl phthalate, glycerol, glyceryl monostearate, glyceryl triacetate, polyoxyethylene/polyoxypropylene copolymers, polyethylene glycol, triethyl citrate, dibutyl phthalate, oils, propylene glycol, and the like, and combinations thereof. The plasticizer may be present in an amount ranging from about 0.5% to about 5% by weight of the composition.

Opacifiers suitable for use in the present invention, include, but are not limited to titanium dioxide, iron oxides, and the like, and combinations thereof. The opacifier may be present in an amount ranging from about 0.1 to about 1% by weight of the composition.

Pore-forming agents for use in the present invention, particularly for inclusion in the extended-release coating, include, but are not limited to, hydrophilic compounds such as silicon dioxide, PVP, HPMC, HPC, lactose, mannitol, PEG, sodium chloride, polysorbate, polyvinyl acetate, gelatin, potassium chloride, sodium laurel sulfate, polyoxyl 40 hydrogenated castor oil, and combinations thereof, and the like. The pore former may be present in an amount ranging from about 0.1 to about 10%. A preferred pore former is amorphous silica sold under the trade name Syloid 244P.

The pharmaceutical compositions as described herein may also contain permitted FD&C dyes and colors.

The pharmaceutical compositions as described herein may be prepared by coating techniques such as spray-coating. For example, inert cores may be coated with a seal coat comprising a binder and, optionally, excipients such as a plasticizer, surfactant, anti-tacking agent and opacifying agent. The components of the seal coat may be dissolved or dispersed in an appropriate solvent and the dispersion may be coated on the inert core in a fluidized bed equipment (such as a Wurster or Glatt). The coated cores may then be dried. A coat of the drug may then be applied to the cores by spraying a suspension or dispersion comprising carvedilol base, an acid component and optionally a binder, in an aqueous or organic solvent, and drying the drug coated cores, thereby removing the solvent. It should be noted that spray drying itself may result in removal of the solvent. Without being bound by any particular theory, spraying a solution of a carvedilol and an acid component, as defined above, on the cores results in in situ formation of amorphous carvedilol salt. The drug-coated cores may optionally be coated with a seal coat or may directly be coated with a coat comprising an extended release composition comprising an extended release polymer. The extended release polymer coat may be applied by dispersing or suspending the extended release polymer in a suitable medium which may additionally comprise excipients, such as a plasticizer, surfactant, anti-tacking agent and opacifying agent, and spraying the resultant dispersion drug-coated cores, followed by drying to obtain extended-release multiparticulate pellets. The cores may optionally be cured by heating at a temperature of about 40° C. to 50° C. for a period of at least about 24 hours. The cores may optionally be mixed with a lubricant and filled into capsules of suitable size or provided as any suitable composition such as tablet or sachet.

Cores of amorphous carvedilol salt may also be provided by providing a solution comprising carvedilol, an acid component and optionally a binder, contacting the solution with at least one pharmaceutically acceptable excipient, and processing the product to obtain cores. For example, carvedilol base, an acid component and a binder or a diluent may be dispersed in an aqueous or organic solvent and sprayed on a mixture of diluent, binder, and optionally a disintegrant in a suitable apparatus, such as a fluidized bed granulator to obtain granules as cores. The granular mass may optionally be mixed with additional quantities of diluent, binder or disintegrant and kneaded with a solvent, and extruded and spheronized to obtain cores comprising amorphous carvedilol phosphate. The cores may then be coated with an extended release composition as described herein.

In a preferred embodiment, an extended-release capsule comprising amorphous carvedilol phosphate may be prepared by
providing one or more inert cores;
coating the core or cores with a dispersion of a binder dissolved in aqueous isopropanol solution;
drying the seal-coated core or cores;
spraying a dispersion of carvedilol base, phosphoric acid, and a binder in an aqueous isopropanol solution over the seal-coated core or cores;
coating the drug-coated core or cores with a dispersion of a binder dissolved in aqueous isopropanol solution;
coating the core or cores with a dispersion comprising an extended-release polymer, binder, plasticizer, opacifier and anti-tacking agent;
optionally curing the core or cores;
optionally mixing the core or cores with a lubricant; and
filling the core or cores into hard gelatin capsules.

In another preferred embodiment, an extended-release capsule comprising amorphous carvedilol phosphate may be prepared by
dispersing carvedilol base, phosphoric acid and a binder or diluent in an aqueous isopropanol solution;
spraying the dispersion on a mixture of diluent, binder, and disintegrant to obtain a granular mass;
mixing the granular mass with additional quantities of diluent, binder or disintegrant in an aqueous isopropanol solution;
extruding and spheronizing the mixture to obtain one or more cores;
coating the core or cores with a dispersion comprising an extended-release polymer, binder, plasticizer, opacifier and anti-tacking agent;
optionally curing the core or cores;
optionally mixing the core or cores with a lubricant; and
filling the core or cores into hard gelatin capsules.

The extended release pharmaceutical compositions as described herein may further comprise an immediate-release portion of carvedilol or a pharmaceutically acceptable salt(s) thereof coated over the extended-release coating. The immediate release portion of carvedilol or a pharmaceutically acceptable salt(s) thereof may also be present in the composition as separate pellets together with extended-release cores filled into hard gelatin capsules or compressed into a tablet. In preferred embodiments, the immediate-release pellets comprise amorphous carvedilol salt, preferably carvedilol phosphate, formed in situ as described herein, but lacking any extended-release coating. The ratio of extended-release pellets to immediate-release pellets can be chosen to provide any desired release in vivo or in vitro profile. For example, the ratio of extended-release pellets to immediate-release pellets can be chosen to provide an in vivo release profile substantially equivalent (e.g., bioequivalent) to COREG® CR, as measured by one or more of Cₘₐₓ, AUC, Tₘₐₓ and T_{1/2}. Alternatively, the ratio of extended-release pellets to immediate-release pellets can be chosen to provide greater bioavailability than that of COREG® CR.

Accordingly, in a preferred embodiment, an extended-release capsule comprising amorphous carvedilol phosphate may be prepared by
providing inert cores;
spraying a dispersion comprising carvedilol base and phosphoric acid over the cores;
drying the drug-coated cores to form a population of immediate-release pellets;
coating a portion of the immediate-release pellets with a dispersion comprising an extended-release polymer to form a population of extended-release pellets;
drying the extended-release pellets; and
filling a portion of the immediate-release pellets and extended-release pellets into hard gelatin capsules.

The extended release pharmaceutical compositions as described herein may contain two different populations of extended-release pellets. Such populations may differ in the type or amount of extended-release coating. The composition may further comprise immediate-release pellets. Again, the ratio of pellets can be chosen to provide any desired release in vivo or in vitro profile.

Accordingly, in a preferred embodiment, an extended-release capsule comprising amorphous carvedilol phosphate may be prepared by
providing inert cores;
spraying a dispersion comprising carvedilol base and phosphoric acid over the cores;
drying the drug-coated cores to form a population of immediate-release pellets;
coating a first portion of the immediate-release pellets with a dispersion comprising a first amount of an extended-release polymer and a first amount of a pore-forming agent to form a first population of extended-release pellets;
coating a second portion of the immediate-release pellets with a dispersion comprising a second amount of an extended-release polymer and a second amount of a pore-forming agent to form a second population of extended-release pellets;
drying the first and second populations of extended-release pellets; and
filling a portion of the immediate-release pellets and first and second populations of extended-release pellets into hard gelatin capsules.

The extended-release polymer and pore-forming agent and their amounts can be the same or different in the two populations of extended-release pellets. In particular embodiments, the amounts of extended-release polymer and pore-forming agent differ between the first and second populations of extended-release pellets. The pore-forming agent forms diffusion pores in the extended-release coating, thereby increasing the rate and extent of release of the drug that would otherwise occur by the coating itself. This improves absorption of the drug along the entire gastro-intestinal tract, particularly when the extended-release polymer is pH-dependent. In this respect, a particularly useful extended-release polymer/pore-forming agent combination is Eudragit L30D55/Syloid 244P. The ratio of extended-release polymer to pore-forming agent is preferably less than about 9:1, 8:1, 7:1 6:1 5:1, 4:1, 3:1 or 2:1 w/w. In some preferred embodiments, the ratio of extended-release polymer to pore-forming agent is about 1:1 w/w.

In alternative embodiments, an extended release tablet comprising amorphous carvedilol phosphate may be prepared by compressing the cores as obtained above into a tablet.

The extended release pharmaceutical compositions as described herein can be used to treat any disease or disorder for which COREG® CR is indicated. For example, the extended release pharmaceutical compositions as described herein can be used to 1) treat mild-to-severe chronic heart failure of ischemic or cardiomyopathic origin, usually in addition to diuretics, ACE inhibitors, and digitalis, to increase survival and, also, to reduce the risk of hospitalization; 2) reduce cardiovascular mortality in clinically stable patients who have survived the acute phase of a myocardial infarction and have a left ventricular ejection fraction of ≦40% (with or without symptomatic heart failure); and 3) manage essential hypertension, alone or in combination with other antihypertensive agents, especially thiazide-type diuretic.

The extended release pharmaceutical compositions as described herein are suitable for one-daily administration and are preferably formulated to contain 10, 20, 40 or 80 mg total amorphous carvedilol salt. In preferred embodiments, the salt is carvedilol phosphate.

The present invention may further be illustrated by the following examples, which are not to be construed as limiting the invention.

### EXAMPLES

### Example 1

### Controlled-Release Carvedilol Phosphate Compositions

| | | |
|---|---|---|
| S/N | Ingredient | % w/w |

Seal coating

| | | |
|---|---|---|
| 1 | Inert non-pareil | 10-90 |
| 2 | Binder | 1-5 |
| 3 | Solvent | q.s. |

Drug layering

| | | |
|---|---|---|
| 4 | Carvedilol | 5-20 |
| 5 | Orthophosphoric acid | 1-10 |
| 6 | Binder | 1-5 |
| 7 | Solvent | q.s. |

Seal coating

| | | |
|---|---|---|
| 8 | Binder | 1-5 |
| 9 | Surfactant | 0-2 |
| 10 | Solvent | q.s. |

Controlled Release Coating

| | | |
|---|---|---|
| 11 | Extended release polymer | 1-10 |
| 12 | Anti-tacking agent | 1-5 |
| 13 | Binder | 1-5 |
| 14 | Plasticizer | 0.5-5 |
| 15 | Solvent | q.s. |

Lubrication

| | | |
|---|---|---|
| 16 | Lubricant | 0.0-2 |

### Procedure

Inert non-pareil of appropriate mesh size are screened and seal-coated with a solution/dispersion of a binder. Carvedilol base, orthophosphoric acid and a binder are dispersed in a solvent and sprayed on the seal-coated non-pareils. The non-pareils coated with amorphous carvedilol phosphate are dried and further coated with a dispersion of binder and optionally a surfactant. Extended release polymer, binder, anti-tacking agent and plasticizer are dispersed in a solvent and coated on the seal-coated drug cores. The cores are optionally cured, mixed with a lubricant and filled in capsules of appropriate size or compressed into a tablet using appropriate tooling.

### Example 2

### Controlled-Release Amorphous Carvedilol Phosphate Composition

| | | | |
|---|---|---|---|
| S/N | Ingredient | mg/Capsule | % w/w |

Seal coating

| | | | |
|---|---|---|---|
| 1 | Sugar spheres | 334.60 | 68.8 |
| 2 | Hydroxypropyl methylcellulose | 13.40 | 2.8 |
| 3 | Isopropyl alcohol | q.s. | — |
| 4 | Purified water | q.s. | — |

Drug layering

| | | | |
|---|---|---|---|
| 5 | Carvedilol | 64.45 | 13.3 |
| 6 | Orthophosphoric acid (88.0% w/w) | 22.43 | 4.6 |
| 7 | Isopropyl alcohol | q.s. | — |
| 8 | Purified water | q.s. | — |
| 9 | Hydroxypropyl methylcellulose | 16.00 | 3.3 |

Seal coating

| | | | |
|---|---|---|---|
| 10 | Hydroxypropyl methylcellulose | 17.00 | 3.5 |
| 11 | Hydrogenated castor oil derivative | 1.00 | 0.2 |
| 12 | Isopropyl alcohol | q.s. | — |
| 13 | Purified water | q.s. | — |

Controlled Release Coating

| | | | |
|---|---|---|---|
| 14 | Polymethacrylate copolymer | 8.50 | 1.7 |
| 15 | Talc | 8.00 | 1.6 |
| 16 | Lactose | 6.00 | 1.2 |
| 17 | Colloidal silicon dioxide | 0.40 | 0.1 |
| 18 | Crospovidone | 0.20 | 0.04 |
| 19 | Purified water | q.s. | — |

Lubrication

| | | | |
|---|---|---|---|
| 20 | Sodium stearyl fumarate | 1.00 | 0.2 |

| | | |
|---|---|---|
| TOTAL | 486.10 | 100.0 |

### Procedure

Inert sugar spheres of appropriate mesh size were screened and coated with a solution containing hydroxypropyl methylcellulose dispersed in a mixture of isopropyl alcohol and water. Carvedilol base, orthophosphoric acid and hydroxypropyl methylcellulose were dispersed in a mixture of isopropyl alcohol and water and sprayed on the seal-coated sugar spheres. The drug-coated spheres were dried and further coated with a dispersion of hydroxypropyl methylcellulose and a castor oil derivative in a mixture of isopropyl alcohol and water. Polymethacrylate copolymer, talc, lactose, colloidal silicon dioxide and crospovidone were dispersed in water coated on the seal-coated drug cores. The cores were optionally cured, mixed with sodium stearyl fumarate and filled in capsules of appropriate size.

### Example 3

### Controlled-Release Amorphous Carvedilol Phosphate Compositions

| | | |
|---|---|---|
| S/N | Ingredients | % w/w |

Core

| | | |
|---|---|---|
| 1 | Carvedilol | 5-30 |
| 2 | Orthophosphoric acid | 1-10 |
| 3 | Diluent | 1-80 |
| 4 | Binder | 1-20 |
| 5 | Disintegrant | 1-5 |
| 6 | Solvent | q.s. |

Coating

| | | |
|---|---|---|
| 7 | Extended Release Polymer | 1-30 |
| 8 | Binder | 1-5 |
| 9 | Anti-tacking agent | 1-10 |
| 10 | Plasticizer | 0.5-5 |
| 11 | Solvent | q.s. |

Lubrication

| | | |
|---|---|---|
| 12 | Lubricant | 0.0-2 |

### Procedure

Carvedilol base, ortho-phosphoric acid and a binder or a diluent are dispersed in a solvent and are sprayed on a mixture of diluent, binder, and optionally a disintegrant in a suitable apparatus, such as a fluidized bed granulator. The granular mass is optionally mixed with additional quantities of diluent, binder or disintegrant and kneaded with a solvent, extruded and spheronized to obtain cores comprising amorphous carvedilol phosphate. The extended release polymer, binder, anti-tacking agent and plasticizer are dispersed in a solvent and coated on the drug cores. The cores are optionally cured, mixed with a lubricant and filled in capsules of appropriate size or compressed into a tablet using appropriate tooling.

### Example 4

### Controlled-Release Amorphous Carvedilol Phosphate Composition

| | | |
|---|---|---|
| S/N | Ingredients | % w/w |

Core

| | | |
|---|---|---|
| 1 | Carvedilol | 5-20 |
| 2 | Orthophosphoric acid | 1-10 |
| 3 | Microcrystalline cellulose | 10-70 |
| 4 | Mannitol | 1-20 |
| 5 | Hydroxypropyl Methylcellulose | 1-10 |
| 6 | Croscarmellose sodium | 1-5 |
| 7 | Isopropyl alcohol | q.s. |
| 8 | Purified water | q.s. |

Coating

| | | |
|---|---|---|
| 8 | Ethylcellulose | 1-30 |
| 9 | Hydroxypropyl methylcellulose | 1-5 |
| 10 | Sodium lauryl sulfate | 0-5 |
| 11 | Triethyl citrate | 0.5-5 |
| 12 | Isopropyl alcohol | q.s. |
| 13 | Dichloromethane | q.s. |

Lubrication

| | | |
|---|---|---|
| 14 | Lubricant | 0.0-2 |

### Procedure

Carvedilol base, ortho-phosphoric acid and hydroxypropyl methylcellulose were dispersed in a mixture of isopropyl alcohol and water and sprayed on a mixture of microcrystalline cellulose, mannitol and croscarmellose sodium by top-spray technique in a fluidized bed granulator. The granular mass was mixed with microcrystalline cellulose, croscarmellose sodium and hydroxypropyl methylcellulose, kneaded with mixture of isopropyl alcohol and water, and extruded and spheronized to obtain cores comprising amorphous carvedilol phosphate. Ethylcellulose, hydroxypropyl methylcellulose, sodium lauryl sulfate and triethyl citrate were dispersed in a mixture of isopropylalcohol and dichloromethane and coated on the drug cores. The cores were optionally cured, mixed with a lubricant and filled in capsules of appropriate size.

### Example 5

### Two-Component Amorphous Carvedilol Phosphate Composition

Carvedilol Phosphate Immediate Release Pellets 10/20/40/80 mg

| | | | 10 mg | | 20 mg | | 40 mg | | 80 mg | |
|---|---|---|---|---|---|---|---|---|---|---|
| | | | mg/ | % | mg/ | % | mg/ | % | mg/ | |
| S/N | Ingredient | Functionality | Cap | w/w | Cap | w/w | Cap | w/w | Cap | % w/w |

Drug Layering

| | | | | | | | | | | |
|---|---|---|---|---|---|---|---|---|---|---|
| 1 | Sugar Spheres | Non Pareil | 29.64 | 76.99 | 59.28 | 76.99 | 118.56 | 76.99 | 237.12 | 76.99 |
| | (Pharma-A- | Seeds (core | | | | | | | | |
| | Spheres 25-30#) | pellets) | | | | | | | | |
| 2 | Carvedilol | API | 5.64 | 14.65 | 11.28 | 14.65 | 22.56 | 14.65 | 45.12 | 14.65 |
| 3 | Orthophosphoric | Salt forming | 1.60 | 4.16 | 3.20 | 4.16 | 6.41 | 4.16 | 12.81 | 4.16 |
| | Acid (85% w/w) | agent | | | | | | | | |
| 4 | Povidone | Binder | 1.35 | 3.50 | 2.70 | 3.50 | 5.39 | 3.50 | 10.78 | 3.50 |
| | (Plasdone K-29/32) | | | | | | | | | |
| 5 | Polyethylene | Solubilizer | 0.22 | 0.56 | 0.43 | 0.56 | 0.87 | 0.56 | 1.74 | 0.56 |
| | Glycol | | | | | | | | | |
| | (Polyglykol400) | | | | | | | | | |
| 6 | Polysorbate 80 | Solubilizer | 0.05 | 0.14 | 0.11 | 0.14 | 0.22 | 0.14 | 0.43 | 0.14 |
| 7 | Isopropyl | Solvent | q.s | — | q.s | — | q.s | — | q.s | — |
| | Alcohol | | | | | | | | | |
| 8 | Purified Water | Solvent | q.s | — | q.s | — | q.s | — | q.s | — |

| | | | | | | | | | |
|---|---|---|---|---|---|---|---|---|---|
| Total | — | 38.50 | 100 | 77.00 | 100 | 154.00 | 100 | 308.00 | 100 |

Carvedilol Phosphate Extended-Release Pellets 10/20/40/80 mg

| | | | 10 mg | | 20 mg | | 40 mg | | 80 mg | |
|---|---|---|---|---|---|---|---|---|---|---|
| | | | mg/ | % | mg/ | % | mg/ | % | mg/ | |
| S/N | Ingredient | Functionality | Cap | w/w | Cap | w/w | Cap | w/w | Cap | % w/w |

Drug Layering

| | | | | | | | | | | |
|---|---|---|---|---|---|---|---|---|---|---|
| 1 | Sugar Spheres | Non Pareil | 12.70 | 70.00 | 25.41 | 70.00 | 50.81 | 70.00 | 101.62 | 70.00 |
| | (Pharma-A- | Seeds (core | | | | | | | | |
| | Spheres25-30#) | pellets) | | | | | | | | |
| 2 | Carvedilol | API | 2.42 | 13.32 | 4.83 | 13.32 | 9.67 | 13.32 | 19.34 | 13.32 |
| 3 | Orthophosphoric | Salt forming | 0.69 | 3.78 | 1.37 | 3.78 | 2.75 | 3.78 | 5.49 | 3.78 |
| | Acid (85% w/w) | agent | | | | | | | | |
| 4 | Povidone | Binder | 0.58 | 3.18 | 1.16 | 3.18 | 2.31 | 3.18 | 4.62 | 3.18 |
| | (Plasdone K-29/32) | | | | | | | | | |
| 5 | Polyethylene | Solubilizer | 0.09 | 0.51 | 0.19 | 0.51 | 0.37 | 0.51 | 0.74 | 0.51 |
| | Glycol | | | | | | | | | |
| | (Polyglykol400) | | | | | | | | | |
| 6 | Polysorbate 80 | Solubilizer | 0.02 | 0.13 | 0.05 | 0.13 | 0.09 | 0.13 | 0.19 | 0.13 |
| 7 | Isopropyl | Solvent | q.s | — | q.s | — | q.s | — | q.s | — |
| | Alcohol | | | | | | | | | |
| 8 | Purified Water | Solvent | q.s | — | q.s | — | q.s | — | q.s | — |

Extended Release Coating

| | | | | | | | | | | |
|---|---|---|---|---|---|---|---|---|---|---|
| 9 | Methacrylic | Control | 0.79 | 4.33 | 1.57 | 4.33 | 3.14 | 4.33 | 6.28 | 4.33 |
| | Acid Copolymer | Release | | | | | | | | |
| | (Eudragit | Polymer | | | | | | | | |
| | L30D55) (Dry | | | | | | | | | |
| | basis) | | | | | | | | | |
| 10 | Silicon Dioxide | Pore former | 0.79 | 4.33 | 1.57 | 4.33 | 3.14 | 4.33 | 6.28 | 4.33 |
| | (Syloid 244FP) | | | | | | | | | |
| 11 | Triethyl Citrate | Plastisizer | 0.08 | 0.43 | 0.16 | 0.43 | 0.31 | 0.43 | 0.62 | 0.43 |
| 12 | Purified Water | Solvent | q.s | — | q.s | — | q.s | — | q.s | — |

| | | | | | | | | | |
|---|---|---|---|---|---|---|---|---|---|
| Total | — | 18.15 | 100 | 36.30 | 100 | 72.59 | 100 | 145.18 | 100 |

### Procedure

### Drug Layering:

Carvedilol was dispersed in isopropyl alcohol and purified water (prewarmed to 45° C.). Orthophosphoric acid was slowly under continuous stirring to produce a clear solution. Povidone, polyethylene glycol and polysorbate 80 were added under continuous stirring to produce a clear drug solution. Stirring was continued for 10 min. Sugar spheres were loaded in a Fluid Bed Multi Technology (FBMT) (bottom spray assembly) and layered with drug solution while maintaining the bed temperature at about 45-50° C. After completion of drug layering, the cores were dried at 40° C. for 1 hour.

### Extended Release Coating:

Silicon dioxide was dispersed in purified water with stirring. Eudragit L30D55 and triethyl citrate were added and stirred for 30 min to produce a uniform dispersion. A portion of the drug-layered cores were loaded in FBMT (bottom spray assembly) and coated with the extended-release dispersion while maintaining the bed temperature at about 35-40° C. After completion of coating, the coated cores were dried at 40° C. for 1 hour.

### Capsules:

Hard gelatin capsules were filled with 70% drug-layered cores (IR pellets) and 30% extended release-coated cores (ER pellets) as below:

| | | 10 mg | | 20 mg | | 40 mg | | 80 mg | |
|---|---|---|---|---|---|---|---|---|---|
| | | mg/ | % | mg/ | % | mg/ | % | mg/ | % |
| S/N | Ingredient | Cap | w/w | Cap | w/w | Cap | w/w | Cap | w/w |
| 1 | Carvedilol Phosphate | 38.50 | 67.96 | 77.00 | 67.96 | 154.00 | 67.96 | 308.00 | 67.96 |
| | IR Pellets | | | | | | | | |
| 2 | Carvedilol Phosphate | 18.15 | 32.04 | 36.30 | 32.04 | 72.60 | 32.04 | 145.20 | 32.04 |
| | ER Pellets | | | | | | | | |
| 3 | Size “4”, Empty Hgc, | 1 No. | — | — | — | — | — | — | — |
| | Green Opaque Cap, | | | | | | | | |
| | White Opaque Body | | | | | | | | |
| | Imprinting-Cap- | | | | | | | | |
| | Amneal, Body-741 | | | | | | | | |
| 4 | Size “3”, Empty Hgc, | — | — | 1 No. | — | — | — | — | — |
| | Yellow Opaque Cap, | | | | | | | | |
| | White Opaque Body | | | | | | | | |
| | Imprinting-Cap- | | | | | | | | |
| | Amneal, Body-742 | | | | | | | | |
| 5 | Size “2”, Empty Hgc, | — | — | — | — | 1 No. | — | — | — |
| | Green Opaque Cap, | | | | | | | | |
| | Yellow Opaque Body | | | | | | | | |
| | Imprinting-Cap- | | | | | | | | |
| | Amneal, Body-743 | | | | | | | | |
| 6 | Size “0”, Empty Hgc, | — | — | — | — | — | — | 1 No. | — |
| | White Opaque Cap, | | | | | | | | |
| | White Opaque Body | | | | | | | | |
| | Imprinting-Cap- | | | | | | | | |
| | Amneal, Body-744 | | | | | | | | |

| | | | | | | | | |
|---|---|---|---|---|---|---|---|---|
| Fill Weight | 56.65 | 100 | 113.30 | 100 | 226.60 | 100 | 453.20 | 100 |

### Example 6

### Three-Component Amorphous Carvedilol Phosphate Composition

Carvedilol Phosphate Immediate-Release Pellets 10/20/40/80 mg

| | | | 10 mg | | 20 mg | | 40 mg | | 80 mg | |
|---|---|---|---|---|---|---|---|---|---|---|
| | | | mg/ | % | mg/ | % | mg/ | % | mg/ | % |
| S/N | Ingredient | Functionality | Cap | w/w | Cap | w/w | Cap | w/w | Cap | w/w |

Drug Layering

| | | | | | | | | | | |
|---|---|---|---|---|---|---|---|---|---|---|
| 1 | Microcrystalline | Non Pareil Seeds | 18.68 | 75.48 | 37.36 | 75.48 | 74.72 | 75.48 | 149.45 | 75.48 |
| | cellulose | (core pellets) | | | | | | | | |
| | spheres | | | | | | | | | |
| | (Celphere CP 203) | | | | | | | | | |
| 2 | Polyethylene | Solubilizer | 0.37 | 1.51 | 0.75 | 1.51 | 1.49 | 1.51 | 2.99 | 1.51 |
| | Glycol 20000 | | | | | | | | | |
| 3 | Dichloromethane | Solvent | q.s | — | q.s | — | q.s | — | q.s | — |
| 4 | Carvedilol | API | 3.63 | 14.65 | 7.25 | 14.65 | 14.50 | 14.65 | 29.01 | 14.65 |
| 5 | Ortho | Salt forming agent | 1.03 | 4.16 | 2.06 | 4.16 | 4.12 | 4.16 | 8.24 | 4.16 |
| | Phosphoric | | | | | | | | | |
| | Acid 85% w/w | | | | | | | | | |
| 6 | Povidone | Binder | 0.87 | 3.50 | 1.73 | 3.50 | 3.47 | 3.50 | 6.93 | 3.50 |
| | (Plasdone K29/32) | | | | | | | | | |
| 7 | Polyethylene | Solubilizer | 0.14 | 0.56 | 0.28 | 0.56 | 0.56 | 0.56 | 1.12 | 0.56 |
| | Glycol | | | | | | | | | |
| | (Polyglykol | | | | | | | | | |
| | 400) | | | | | | | | | |
| 8 | Polysorbate 80 | Solubilizer | 0.03 | 0.14 | 0.07 | 0.14 | 0.14 | 0.14 | 0.28 | 0.14 |
| 9 | Isopropyl | Solvent | q.s | — | q.s | — | q.s | — | q.s | — |
| | Alcohol | | | | | | | | | |
| 10 | Purified Water | Solvent | q.s | — | q.s | — | q.s | — | q.s | — |

| | | | | | | | | |
|---|---|---|---|---|---|---|---|---|
| Total | 24.75 | 100 | 49.50 | 100 | 99.00 | 100 | 198.00 | 100 |

Carvedilol Phosphate Extended-Release Pellets-1 10/20/40/80 mg

| | | | 10 mg | | 20 mg | | 40 mg | | 80 mg | |
|---|---|---|---|---|---|---|---|---|---|---|
| | | | mg/ | % | mg/ | % | mg/ | % | mg/ | % |
| S/N | Ingredient | Functionality | Cap | w/w | Cap | w/w | Cap | w/w | Cap | w/w |

Drug Layering

| | | | | | | | | | | |
|---|---|---|---|---|---|---|---|---|---|---|
| 1 | Microcrystalline | Non Pareil | 6.23 | 74.00 | 12.45 | 74.00 | 24.91 | 74.00 | 49.82 | 74.00 |
| | cellulose | Seeds (core | | | | | | | | |
| | spheres | pellets) | | | | | | | | |
| | (Celphere CP 203) | | | | | | | | | |
| 2 | Polyethylene | Solubilizer | 0.12 | 1.48 | 0.25 | 1.48 | 0.50 | 1.48 | 1.00 | 1.48 |
| | Glycol 20000 | | | | | | | | | |
| 3 | Dichloromethane | Solvent | q.s | — | q.s | — | q.s | — | q.s | — |
| 4 | Carvedilol | API | 1.21 | 14.36 | 2.42 | 14.36 | 4.83 | 14.36 | 9.67 | 14.36 |
| 5 | Ortho | Salt forming | 0.34 | 4.08 | 0.69 | 4.08 | 1.37 | 4.08 | 2.75 | 4.08 |
| | Phosphoric | agent | | | | | | | | |
| | Acid 85% w/w | | | | | | | | | |
| 6 | Povidone | Binder | 0.29 | 3.43 | 0.58 | 3.43 | 1.16 | 3.43 | 2.31 | 3.43 |
| | (Plasdone K29/32) | | | | | | | | | |
| 7 | Polyethylene | Solubilizer | 0.05 | 0.55 | 0.09 | 0.55 | 0.19 | 0.55 | 0.37 | 0.55 |
| | Glycol | | | | | | | | | |
| | (Polyglykol | | | | | | | | | |
| | 400) | | | | | | | | | |
| 8 | Polysorbate 80 | Solubilizer | 0.01 | 0.14 | 0.02 | 0.14 | 0.05 | 0.14 | 0.09 | 0.14 |
| 9 | Iso Propyl | Solvent | q.s | — | q.s | — | q.s | — | q.s | — |
| | Alcohol | | | | | | | | | |
| 10 | Purified Water | Solvent | q.s | — | q.s | — | q.s | — | q.s | — |

Functional Coating

| | | | | | | | | | | |
|---|---|---|---|---|---|---|---|---|---|---|
| 11 | Methacrylic | Control | 0.08 | 0.93 | 0.16 | 0.93 | 0.31 | 0.93 | 0.63 | 0.93 |
| | Acid | Release | | | | | | | | |
| | Copolymer | Polymer | | | | | | | | |
| | (Eudragit | | | | | | | | | |
| | L30D55) (Dry | | | | | | | | | |
| | basis) | | | | | | | | | |
| 12 | Silicon | Poreformer | 0.08 | 0.93 | 0.16 | 0.93 | 0.31 | 0.93 | 0.63 | 0.93 |
| | Dioxide | | | | | | | | | |
| | (Syloid 244FP) | | | | | | | | | |
| 13 | Triethyl | Plastisizer | 0.01 | 0.09 | 0.02 | 0.09 | 0.03 | 0.09 | 0.06 | 0.09 |
| | Citrate | | | | | | | | | |
| 14 | Purified Water | Solvent | q.s | — | q.s | — | q.s | — | q.s | — |

| | | | | | | | | |
|---|---|---|---|---|---|---|---|---|
| Total | 8.41 | 100 | 16.83 | 100 | 33.66 | 100 | 67.32 | 100 |

Carvedilol Phosphate Extended-Release Pellets-2 10/20/40/80 mg

| | | | 10 mg | | 20 mg | | 40 mg | | 80 mg | |
|---|---|---|---|---|---|---|---|---|---|---|
| | | | mg/ | % | mg/ | % | mg/ | % | mg/ | % |
| S/N | Ingredient | Functionality | Cap | w/w | Cap | w/w | Cap | w/w | Cap | w/w |

Drug Layering

| | | | | | | | | | | |
|---|---|---|---|---|---|---|---|---|---|---|
| 1 | Macrocrystalline | Non Pareil | 16.61 | 68.62 | 33.21 | 68.62 | 66.42 | 68.62 | 132.84 | 68.62 |
| | cellulose spheres | Seeds | | | | | | | | |
| | (Celphere CP 203) | (core pellets) | | | | | | | | |
| 2 | Polyethylene | Solubilizer | 0.33 | 1.37 | 0.66 | 1.37 | 1.33 | 1.37 | 2.66 | 1.37 |
| | Glycol 20000 | | | | | | | | | |
| 3 | Dichloromethane | Solvent | q.s | 0.00 | q.s | 0.00 | | 0.00 | q.s | 0.00 |
| 4 | Carvedilol | API | 3.22 | 13.32 | 6.45 | 13.32 | 12.89 | 13.32 | 25.78 | 13.32 |
| 5 | Ortho | Salt forming | 0.92 | 3.78 | 1.83 | 3.78 | 3.66 | 3.78 | 7.32 | 3.78 |
| | Phosphoric Acid | agent | | | | | | | | |
| | 85% w/w | | | | | | | | | |
| 6 | Povidone | Binder | 0.77 | 3.18 | 1.54 | 3.18 | 3.08 | 3.18 | 6.16 | 3.18 |
| | (Plasdone K29/32) | | | | | | | | | |
| 7 | Polyethylene | Solubilizer | 0.12 | 0.51 | 0.25 | 0.51 | 0.50 | 0.51 | 0.99 | 0.51 |
| | Glycol | | | | | | | | | |
| | (Polyglykol400) | | | | | | | | | |
| 8 | Polysorbate 80 | Solubilizer | 0.03 | 0.13 | 0.06 | 0.13 | 0.12 | 0.13 | 0.25 | 0.13 |
| 9 | Iso Propyl | Solvent | q.s | — | q.s | — | q.s | — | q.s | — |
| | Alcohol | | | | | | | | | |
| 10 | Purified Water | Solvent | q.s | — | q.s | — | q.s | — | q.s | — |

Functional Coating

| | | | | | | | | | | |
|---|---|---|---|---|---|---|---|---|---|---|
| 11 | Methacrylic | Control | 1.05 | 4.33 | 2.09 | 4.33 | 4.19 | 4.33 | 8.38 | 4.33 |
| | Acid | Release | | | | | | | | |
| | Copolymer | Polymer | | | | | | | | |
| | (Eudragit | | | | | | | | | |
| | L30D55) (Dry | | | | | | | | | |
| | basis) | | | | | | | | | |
| 12 | Silicon Dioxide | Poreformer | 1.05 | 4.33 | 2.09 | 4.33 | 4.19 | 4.33 | 8.38 | 4.33 |
| | (Syloid 244FP) | | | | | | | | | |
| 13 | Triethyl Citrate | Plastisizer | 0.11 | 0.43 | 0.21 | 0.43 | 0.42 | 0.43 | 0.84 | 0.43 |
| 14 | Purified Water | Solvent | q.s | — | q.s | — | q.s | — | q.s | — |

| | | | | | | | | |
|---|---|---|---|---|---|---|---|---|
| Total | 24.20 | 100 | 48.40 | 100 | 96.80 | 100 | 193.59 | 100 |

### Procedure

### Seal Coating:

Polyethylene glycol 20000 was added to dichloromethane and stirred for 30 min to produce a clear seal coat solution. Microcrystalline cellulose spheres were loaded in FBMT (bottom spray assembly) and sprayed with seal coat solution while maintaining the bed temperature at about 30-35° C. After completion of seal coating, the pellets were dried for 30 min at 40° C.

### Drug Layering:

Carvedilol was dispersed in isopropyl alcohol and purified water (prewarmed to 45° C.). Orthophosphoric acid was slowly under continuous stirring to produce a clear solution. Povidone, polyethylene glycol and polysorbate 80 were added under continuous stirring to produce a clear drug solution. Stirring was continued for 10 min. Seal-coated microcrystalline cellulose spheres were loaded in FBMT (bottom spray assembly) and layered with drug solution while maintaining the bed temperature at about 45-50° C. After completion of drug layering, the cores were dried at 40° C. for 1 hour.

### Extended Release Coating:

Silicon dioxide was dispersed in purified water with stirring. Eudragit L30D55 and triethyl citrate were added and stirred for 30 min to produce a uniform dispersion. A portion of the drug-layered cores were loaded in FBMT (bottom spray assembly) and coated to a level of 2% with the extended-release dispersion while maintaining the bed temperature at about 35-40° C. Another portion of the drug-layer cores was coated to a level of 10%. After completion of coating, the coated cores were dried at 40° C. for 1 hour.

### Capsules:

Hard gelatin capsules were filled with about 43% drug-layered cores (IR pellets), about 15% of 2%-coated extended-release cores (ER-1 pellets) and about 42% of 10%-coated extended-release cores (ER-2 pellets) as below:

| | | 10 mg | | 20 mg | | 40 mg | | 80 mg | |
|---|---|---|---|---|---|---|---|---|---|
| | | mg/ | % | mg/ | % | mg/ | % | mg/ | % |
| S/N | Ingredient | Cap | w/w | Cap | w/w | Cap | w/w | Cap | w/w |
| 1 | Carvedilol Phosphate | 24.75 | 43.15 | 49.50 | 43.15 | 99.00 | 43.15 | 198.00 | 43.15 |
| | IR Pellets | | | | | | | | |
| 2 | Carvedilol Phosphate | 8.41 | 14.66 | 16.82 | 14.66 | 33.64 | 14.66 | 67.28 | 14.66 |
| | ER-1 Pellets | | | | | | | | |
| 3 | Carvedilol Phosphate | 24.20 | 42.19 | 48.40 | 42.19 | 96.80 | 42.19 | 193.60 | 42.19 |
| | ER-2 Pellets | | | | | | | | |
| 3 | Size “4”, Empty Hgc, | 1 No. | — | — | — | — | — | — | — |
| | Green Opaque Cap, | | | | | | | | |
| | White Opaque Body | | | | | | | | |
| | Imprinting-Cap- | | | | | | | | |
| | Amneal, Body-741 | | | | | | | | |
| 4 | Size “3”, Empty Hgc, | — | — | 1 No. | — | — | — | — | — |
| | Yellow Opaque Cap, | | | | | | | | |
| | White Opaque Body | | | | | | | | |
| | Imprinting-Cap- | | | | | | | | |
| | Amneal, Body-742 | | | | | | | | |
| 5 | Size “2”, Empty Hgc, | — | — | — | — | 1 No. | — | — | — |
| | Green Opaque Cap, | | | | | | | | |
| | Yellow Opaque Body | | | | | | | | |
| | Imprinting-Cap- | | | | | | | | |
| | Amneal, Body-743 | | | | | | | | |
| 6 | Size “0”, Empty Hgc, | — | — | — | — | — | — | 1 No. | — |
| | White Opaque Cap, | | | | | | | | |
| | White Opaque Body | | | | | | | | |
| | Imprinting-Cap- | | | | | | | | |
| | Amneal, Body-744 | | | | | | | | |

| | | | | | | | | |
|---|---|---|---|---|---|---|---|---|
| Fill Weight | 57.36 | 100 | 114.72 | 100 | 229.44 | 100 | 458.88 | 100 |

### Example 7

### Controlled-Release Amorphous Carvedilol Phosphate Composition

| | | 743-PD050 |
|---|---|---|
| Sr. No. | Ingredients | (mg/capsule) |
| 1 | Celphere CP 203 (non periel seeds) | 135.32 |
| 2 | PEG 20000P (solubilizer) | 3.3 |
| 3 | Dichloromethane | q.s. |

Drug layering

| | | |
|---|---|---|
| 4 | Carvedilol (base) | 32.23 |
| 5 | Orthophosphoric Acid | 9.15 |
| 6 | Sodium Lauryl sulphate (solubilizer) | 5.0 |
| 7 | Plasdone K29/32 (binder) | 16.0 |
| 8 | Polyplasdone XL 10 (disintegrant) | 21.0 |
| 9 | Isopropyl alcohol | q.s. |
| 10 | Purified water | q.s. |

Control coat

| | | |
|---|---|---|
| 11 | Drug layered pellets | 222.0 |
| 12 | Eudragit L30D55 (release controller) | 2.11 |
| 13 | Syloid 244FP (pore former) | 2.11 |
| 14 | Triethylcitrate | 0.21 |
| 15 | Purified water | q.s. |

| | |
|---|---|
| Total | 255.30 |

Amorphous carvedilol phosphate (40 mg) pellets were prepared and coated with an extended-release coating as described above. The in vitro dissolution profile of the pellets in phosphate buffer, pH 6.0 with 0.25% SLS/900 mL/paddle/50 RPM was compared with that of COREG® CR pellets (40 mg). The following profiles were obtained:

| | | Coreg CR | 743-PD050A |
|---|---|---|---|
| Sr. No. | Time (hr) | 9ZP3662 (40 mg) | 40 mg |
| 1 | 0.5 | 9.2 | 25 |
| 2 | 1 | 28.0 | 40 |
| 3 | 2 | 46.3 | 53 |
| 4 | 4 | 65.4 | 74 |
| 5 | 6 | 82.0 | 80 |
| 6 | 8 | 90.7 | 87 |
| 7 | 10 | 99.3 | 92 |
| 8 | 12 | 101.7 | 97 |

What is claimed:

1. A core comprising a first coating of an amorphous carvedilol salt, wherein the amorphous carvedilol salt is formed in situ during coating of the core.

2. A pharmaceutical composition comprising a plurality of cores of claim 1 and one or more pharmaceutically acceptable excipients.

3. The pharmaceutical composition of claim 2, wherein the carvedilol salt is carvedilol phosphate.

4. The pharmaceutical composition of claim 3, in capsule form.

5. The pharmaceutical composition of claim 4, wherein the capsule contains 10, 20, 40 or 80 mg total carvedilol phosphate.

6. The pharmaceutical composition of claim 5, wherein at least a portion of the cores comprise a second coating on the first coating comprising an extended-release polymer.

7. The pharmaceutical composition of claim 6, wherein the extended-release polymer is an ethyl acrylate-methyl methacrylate co-polymer.

8. The pharmaceutical composition of claim 7, wherein the second coating comprises a pore-forming agent.

9. The pharmaceutical composition of claim 8, wherein the pore-forming agent is amorphous silica.

10. The pharmaceutical composition of claim 6, wherein at least a portion of the cores lack an extended-release coating.

11. The pharmaceutical composition of claim 10, wherein the portion of the cores comprising a second coating consists of cores comprising different amounts of extended-release polymer and/or pore forming agent.

12. A process for the preparation of a core comprising a first coating of an amorphous carvedilol salt, wherein the carvedilol salt is formed in situ during application of the coating, comprising:
a. providing one or more inert cores;
b. contacting the inert cores with a solution or a dispersion comprising carvedilol, an acid component and optionally a binder, in a solvent; and
c. removing the solvent.

13. The process of claim 12, wherein the acid component is phosphoric acid.

14. The process of claim 13, further comprising coating at least a portion of the cores with a second coating comprising an extended-release polymer.

15. The process of claim 14, wherein the second coating comprises a plasticizer and a pore-forming agent.

16. The process of claim 15, wherein the extended-release polymer is an ethyl acrylate-methyl methacrylate co-polymer and the pore-forming agent is amorphous silica.

17. The process of claim 16, further comprising filling the cores into a gelatin capsule.

18. A core made by the process of claim 12.

19. A method of treating heart failure comprising administering the pharmaceutical composition of claim 10 to a patient in need thereof.

20. A process for preparing an extended-release capsule comprising amorphous carvedilol phosphate, comprising:
a. providing one or more inert cores;
b. spraying a dispersion of carvedilol base, phosphoric acid, and a binder in a solvent over the cores;
c. removing the solvent such that amorphous carvedilol phosphate is formed;
d. coating the drug-coated cores with a dispersion comprising an extended-release polymer, plasticizer, and a pore-forming agent; and
e. filling the cores into a gelatin capsules.
